(19) United States
(12) Patent Application Publication
(10) Pub. No.: US 2012/0029518 A1
(43) Pub. Date: Feb. 2, 2012

(54) LUMBO-SACRAL IMPLANT SYSTEM AND METHOD

(75) Inventors: Jonathan E. Blackwell, Arlington, TN (US); Anthony J. Melkent, Memphis, TN (US); Lindsey Waugh, Memphis, TN (US); Sarah Nicole Johnson, Murfreesboro, TN (US)

(73) Assignee: WARSAW ORTHOPEDIC, INC, Warsaw, IN (US)

(21) Appl. No.: 12/845,828

(22) Filed: Jul. 29, 2010

(51) Int. Cl. A61B 17/16; A61B 17/56; A61F 2/44; A61B 1/32

(52) U.S. Cl. 606/79; 600/201; 606/99; 623/17.11

(57) ABSTRACT

A system for treating a lumbo-sacral junction includes at least one dilator configured to create a passageway from a direct lateral trajectory in a body defining a longitudinal axis. A cutting instrument is configured to create a bore, oriented at an angle from the longitudinal axis, in a L5 vertebrae of the body extending to a central portion of a L5-S1 intervertebral disc space of the body. A delivery instrument is configured to deliver an implant through the bore to the central portion of the L5-S1 disc space. Methods of use are disclosed.

### TECHNICAL FIELD

The present disclosure generally relates to medical devices for the treatment of musculoskeletal disorders, and more particularly to an implant system and method for treating a lumbo-sacral region of a vertebral column.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. Fusion and fixation treatment may employ implants such as interbody fusion devices to achieve arthrodesis. This disclosure describes an improvement over these prior art technologies.

### SUMMARY OF THE INVENTION

Accordingly, an implant system and method is provided for treating the lumbo-sacral region of a vertebral column. It is contemplated that the system and method may be employed for an arthrodesis treatment. It is further contemplated that the implant system and method may be employed for an L5-S1 fusion through a direct lateral interbody trajectory.

In one particular embodiment, in accordance with the principles of the present disclosure, a system for treating a lumbo-sacral region is provided. The system includes at least one dilator configured to create a passageway from a direct lateral trajectory in a body defining a longitudinal axis. A cutting instrument is configured to create a bore, oriented at an angle from the longitudinal axis, in a L5 vertebrae of the body extending to a central portion of a L5-S1 intervertebral disc space of the body. A delivery instrument is configured to deliver an implant through the bore to the central portion of the L5-S1 disc space.

In one embodiment, a method for treating a lumbo-sacral region is provided. The method includes the steps of: making an incision in a lateral portion of a body, the body defining a longitudinal axis; creating a passageway extending from the incision to adjacent a L4-L5 intervertebral disc space of the body, the passageway being disposed at a first angle from the longitudinal axis; creating a bore in a L5 vertebrae of the body, the bore extending at a second angle from the longitudinal axis from the passageway to a central portion of a L5-S1 intervertebral disc space of the body; preparing the L5-S1 intervertebral disc space; and delivering an implant through the bore to the central portion of the L5-S1 intervertebral disc space for treatment.

In one embodiment, a system for treating a lumbo-sacral region employing a direct lateral interbody fusion approach is provided. The system includes a plurality of dilators configured to create a passageway through a direct lateral trajectory in a body defining a longitudinal axis. The passageway is disposed at a first angle from the longitudinal axis. A retractor defines a transverse axis and is connected with the passageway. The retractor is configured for mounting with the body such that the transverse axis is disposed at a non-parallel orientation with the longitudinal axis. A cutting instrument is configured to create a bore, extending at a second angle from the longitudinal axis, from the passageway to a central portion of a L5-S1 intervertebral disc space of the body. A delivery instrument includes a driver connected to a distal end thereof. An implant defines a socket that receives the driver and is engaged thereby such that the implant is deliverable along the second angle of the bore and rotatable relative to the bore into the central portion of an L5-S1 intervertebral disc space.

### BRIEF DESCRIPTION OF THE DRAWINGS

The present disclosure will become more readily apparent from the specific description accompanied by the following drawings, in which:

FIG. 1 is a plan view, in part cross section, of a lumbo-sacral region of a body and one particular embodiment of an implant system in accordance with the principles of the present disclosure;

FIG. 2 is a side view of the region and the implant system shown in FIG. 1;

FIG. 3 is a plan view, in part cross section, of the region and the implant system shown in FIG. 1;

FIG. 4 is an enlarged plan view of component parts of the implant system shown in FIG. 3;

FIG. 5 is a plan view, in part cross section, of the region and one embodiment of the implant system shown in FIG. 3;

FIG. 6 is a plan view, in part cross section, of the region and one embodiment of the implant system shown in FIG. 3;

FIG. 7 is a side view of the region and the implant system shown in FIG. 6;

FIG. 8 is a plan view, in part cross section, of the region and one embodiment of the implant system shown in FIG. 3; and

FIG. 9 is a side view of the region and the implant system shown in FIG. 8.

Like reference numerals indicate similar parts throughout the figures.

### DETAILED DESCRIPTION OF THE INVENTION

The exemplary embodiments of the implant system and related methods of use disclosed are discussed in terms of medical devices for the treatment of musculoskeletal disorders and more particularly, in terms of an implant system and method for treating a lumbo-sacral region of a vertebral column. It is envisioned that the implant system and methods of use disclosed provide a reliable and safe access to a lumbo-sacral region to perform a direct lateral interbody surgical procedure. It is further envisioned that the implant system is configured to deliver an implant to the lumbo-sacral region for an arthrodesis treatment, such as, for example, fusion and fixation.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed implant system may be alternatively employed in a surgical treatment with a patient in a prone or supine position, and/or employ various surgical approaches to the spine, including anterior, posterior, posterior mid-line, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

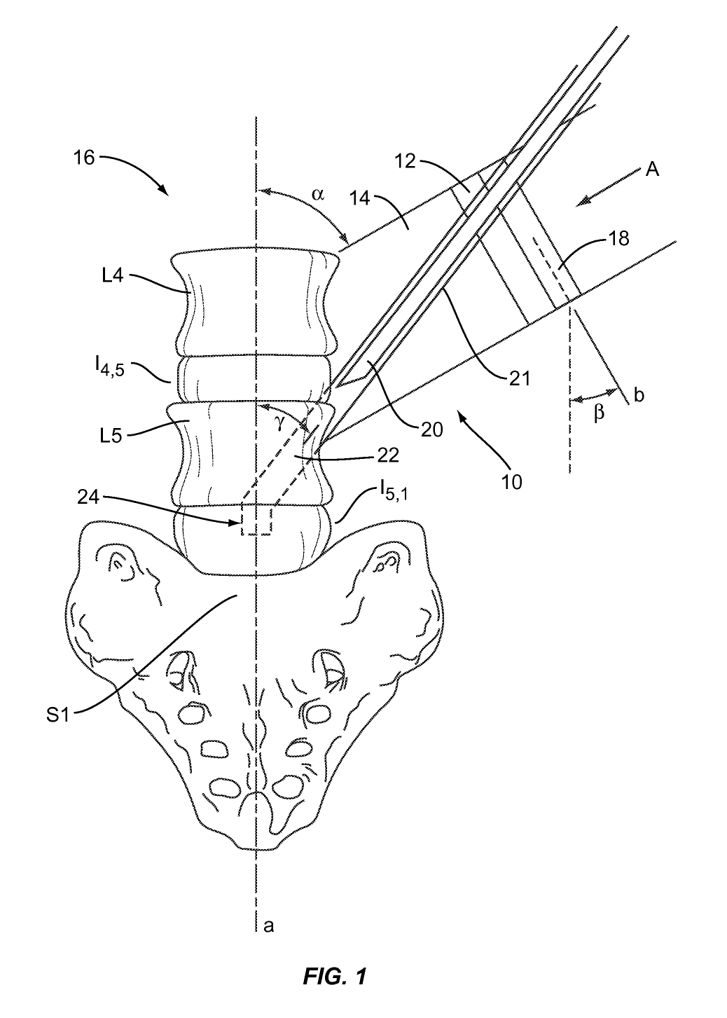

The following discussion includes a description of an implant system and related methods of employing the implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-4, there is illustrated components of an implant system 10 in accordance with the principles of the present disclosure.

The components of implant system 10 are fabricated from materials suitable for medical applications, including metals, polymers, ceramics, biocompatible materials, bone, autograft, allograft and/or their composites, depending on the particular application and/or preference of a medical practitioner. For example, the components of the surgical assembly, individually or collectively, can be fabricated from materials such as stainless steel, titanium, thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, biocompatible materials such as polymers including plastics, metals, ceramics and composites thereof, rigid polymers including polyphenylene, polyimide, polyimide, polyetherimide, polyethylene, epoxy, and various components of the implant system, may have material composites, including the above materials, to achieve various desired characteristics such as strength, rigidity, elasticity, compliance, biomechanical performance, durability and radiolucency or imaging preference.

Implant system 10 is configured for treating a lumbo-sacral region in a surgical fusion procedure from a direct lateral interbody trajectory for stabilizing a L5-S1 junction of a body 16 of a patient. Implant system 10 includes a dilator 12 configured to create a passageway 14. Passageway 14 is oriented from a direct lateral trajectory as shown by arrow A in FIG. 1 in body 16, which defines a longitudinal axis a. One or a plurality of dilators 12 may be used.

Passageway 14 extends from an incision (not shown) made in body 16, to adjacent a L4-L5 intervertebral disc space I_{4,5} of body 16. Passageway 14 is disposed at a first angle α from longitudinal axis a. Dilator 12 is configured for insertion with the incision created in a lateral portion of body 16 in a direct lateral interbody surgical approach. It is contemplated that first angle α may be of various measure according to the requirements of a particular surgical application. It is further contemplated that passageway 14 may be orthogonal to longitudinal axis a.

Dilator 12 is employed with a retractor 18 defining a transverse axis b. Retractor 18 is connected with passageway 14 and configured for mounting with body 16 such that transverse axis b is disposed at a non-parallel orientation with longitudinal axis a. Retractor 18 is positioned to spread apart the incision leading to passageway 14 and maintain passageway 14 in an open orientation adjacent to the outer surface of body 16. Retractor 18 is docked at angle β, as shown in FIG. 1, for approximate alignment with disc space I_{4,5}. Angle β corresponds to the relative angular orientation of axis b with respect to an axis (shown in phantom in FIG. 1) that is parallel to axis a. Angle β is oriented such that instrumentation can be inserted through a L5 vertebrae of body 16 and enter a central portion of a L5-S1 intervertebral disc space I_{5,1}. It is contemplated that angle β may be of various measure according to the requirements of a particular surgical application. It is further contemplated that retractor 18 may include and/or be included with dilator 12 as one component, or may be separate components of implant system 10.

In one embodiment, implant system 10 may be employed with a percutaneous approach. For example, the at least one dilator may include a cannula, mini-open retractor or tube, similar to sleeve 21 discussed below, which creates and defines a passageway for passage of the components of implant system 10, discussed herein, to a surgical site from a direct lateral trajectory, as discussed herein. It is contemplated that this percutaneous embodiment only employs the at least one dilator, such as sleeve 21, and does not utilize dilator 12, passageway 14 and retractor 18.

Implant system 10 includes a cutting instrument 20 configured to create a bore 22 in the L5 vertebrae. Cutting instrument 20 is delivered through passageway 14 and to a surgical site via a sleeve 21 disposed within passageway 14. Bore 22 is oriented at an angle γ from longitudinal axis a. Bore 22 extends to a central portion 24 of disc space I_{5,1}. It is contemplated that angle γ may be in a range of 0-80 degrees to facilitate creation of bore 22 through an upper edge of the L5 vertebrae and extend to central portion 24, although other ranges are envisioned. It is further contemplated that bore 22 may be variously sized and configured, such as, for example, circular, oval, polygonal cross section, uniform diameter, non-uniform diameter, offset and/or arcuate portions, according to the requirements of a particular surgical application.

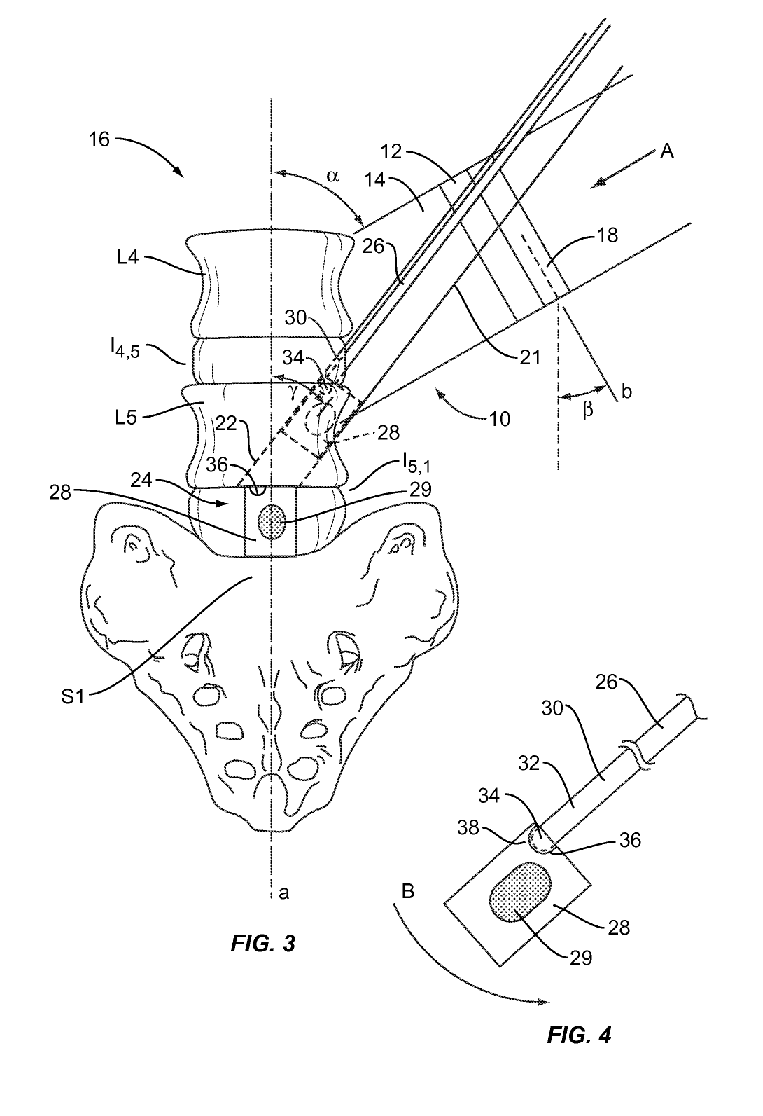

Implant system 10 includes a delivery instrument 26, as shown in FIGS. 3-4, configured to deliver an implant 28 through bore 22 to central portion 24 of disc space I_{5,1}. Delivery instrument 26 is delivered through passageway 14 and to the surgical site via sleeve 21. Delivery instrument 26 includes a driver 30 disposed at a distal end 32. Driver 30 is configured to engage implant 28 such that implant 28 is deliverable along angle γ of bore 22 and rotatable relative to bore 22 into central portion 24 of disc space I_{5,1}.

Driver 30 has a ball configuration, which includes a ball tipped tamp 34. Implant 28 defines a socket 36 configured to receive tamp 34 such that implant 28 is deliverable and rotatable. The ball tamp 34 and socket 36 configuration facilitates manipulating and rotation of implant 28 in a counter clockwise direction, as shown by arrow B in FIG. 4, about the turn from bore 22 into central portion 24. Socket 36 is disposed at a side 38 of implant 28, such as, for example, the upper left corner of implant 28 so that impaction with ball tamp 34 turns implant 28 into disc space I_{5,1}. It is contemplated that implant 28 may rotate in a clockwise direction, and/or be pivoted laterally for manipulation into central portion 24 of disc space I_{5,1}. It is further contemplated that distal end 32 may be flat or planar, smooth, rough, textured and/or detachably mounted with implant 28.

In assembly, operation and use, implant system 10 is employed, for example, with a minimally invasive surgical procedure with a patient from a direct lateral interbody trajectory, such as, for example, a direct lateral fusion procedure for stabilizing the L5-S1 junction of body 16. In one embodiment, implant system 10 is employed to insert implant 28 within disc space I_{5,1} to space apart articular joint surfaces, provide support and maximize stabilization of the L5-S1 junction. It is contemplated that implant 28 may include a metal, plastic and/or bone spacer. It is further contemplated that implant 28 may be variously sized and dimensioned, such as, for example, round, rectangular, trapezoidal, arcuate surfaced, elliptical, smooth surfaced, textured, serrated and/or undulating. It is envisioned that implant system 10 may be used in any existing surgical method or technique including open surgery, mini-open surgery, minimally invasive surgery and percutaneous surgical implantation. Implant 28 can be delivered or implanted as a pre-assembled device or can be assembled in situ. The implant system may be completely or partially revised, removed or replaced in situ.

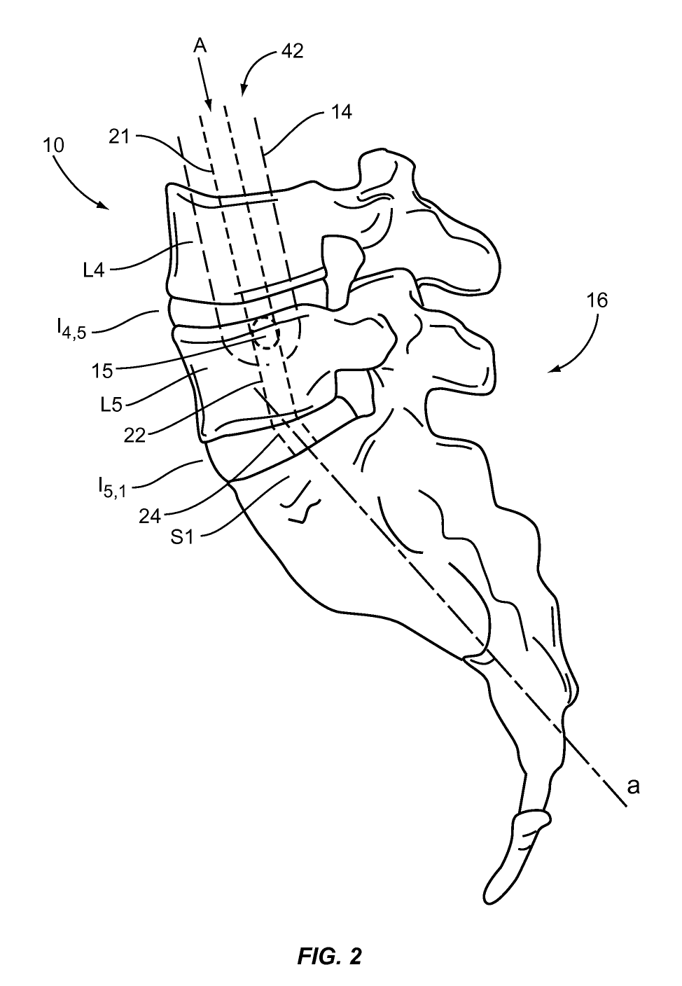

For example, during a direct lateral interbody fusion procedure, a surgeon will make an incision in the skin of lateral portion 42 (FIG. 2) of a patient over and in approximate alignment with disc space I_{4,5}. Dilator 12 is employed to separate the muscles and tissues to create passageway 14 through which the surgery may be performed. It is contemplated that dilator 12 may include one or a plurality of dilators to gradually separate muscle and tissue to create a portal including passageway 14. It is further contemplated that dilator 12 may be configured as an in-situ guidance instrument and may include an endoscope camera tip.

Retractor 18 is positioned and docked at angle β, as discussed above, adjacent the surgical site over the incision. It is envisioned that retractor 18 may be positioned, repositioned and/or adjusted, to one or a plurality of orientations.

Passageway 14 is created and extends from the incision to approximately adjacent disc space I_{4,5}. Passageway 14 is disposed at angle α from longitudinal axis a. Cutting instrument 20 is inserted within passageway 14 via sleeve 21 and creates bore 22 in the L5 vertebrae. It is envisioned that cutting instrument 20 may include a drill, trephine or reamer. Bore 22 extends at angle α from the initial intersection of passageway 14 and the L5 vertebrae that includes an opening 15 adjacent the upper edge of the L5 vertebrae, to a lower endplate of the L5 vertebrae at central portion 24 of disc space I_{5,1}. Cutting instrument 20 is removed from passageway 14 thereafter.

A preparation instrument(s) (not shown) is inserted within passageway 14 and disposed within disc space I_{5,1}. It is envisioned that the preparation instrument(s) may include rasps, curettes and/or a rotating tissue remover such as a rapid disc removal system that can be low profile to cut and remove disc and/or bone material simultaneously. The preparation instrument(s) is employed to remove disc tissue and fluids, adjacent tissues and/or bone, scrape and/or remove tissue from endplate surfaces of the lumbo-sacral region, as well as for aspiration and irrigation of the region according to the requirements of a particular surgical application. The preparation instrument is removed from passageway 14 thereafter.

Delivery instrument 26 is inserted within passageway 14 via sleeve 21 to deliver implant 28 through bore 22 to central portion 24 and into disc space I_{5,1} for the arthrodesis treatment. It is envisioned that the delivery instrument 26 may include a threaded inserter or grasping instrument. Implant 28 is releasably mounted with distal end 32 of driver 30. Driver 30 delivers implant 28 through passageway 14 into bore 22. Implant 28 is passed through bore 22 to adjacent central portion 24 and the lower endplate of the L5 vertebrae.

Ball tamp 34 engages socket 36 of implant 28, as discussed above, to manipulate and rotate central portion 24. As shown by arrow B, in FIG. 4, ball tamp 34 engages socket 36 to pivot implant 28 in rotation about bore 22 and the lower endplate opening of bore 22 such that implant 28 becomes disposed in the prepared disc space I_{5,1}. Delivery instrument 26 manipulates and orients implant 28 within disc space I_{5,1} according to the requirements of a particular surgical application.

Implant 28 is manipulated to engage opposing endplates of the L5 and S1 vertebrae. The surface of implant 28 engages and spaces apart the opposing endplates and is secured within disc space I_{5,1} to stabilize and immobilize portions of the lumbo-sacral junction. Fixation of implant 28 with the endplate surfaces may be facilitated by the resistance provided by the joint space and/or engagement with the endplate surfaces. It is contemplated that implant 28 may engage only one endplate. Delivery instrument 26 is removed from passageway 14 thereafter.

In one embodiment, implant 28 may include fastening elements, which may include locking structure, configured for fixation with the endplate surfaces to secure joint surfaces and provide complementary stabilization and immobilization to the lumbo-sacral region. It is envisioned that locking structure may include fastening elements such as, for example, clips, hooks, adhesives and/or flanges. It is envisioned that implant system 10 can be used with screws to enhance fixation, as described below. It is contemplated that implant system 10 and any screws and attachments may be coated with an osteoconductive material such as hydroxyapatite and/or osteoinductive agent such as a bone morphogenic protein for enhanced bony fixation to the treated area. Implant system 10 can be made of radiolucent materials such as polymers. Radiomarkers may be included for identification under x-ray, fluoroscopy, CT or other imaging techniques.

In one embodiment, implant 28 may include voids and/or openings, such as, for example, a cavity 29 (FIGS. 3-4) for including therapeutic polynucleotides or polypeptides and bone growth promoting material, which can be packed or otherwise disposed therein. It is contemplated that cavity 29 includes lateral openings (FIG. 3) oriented and facing disc space I_{5,1}, and longitudinal openings (not shown) oriented and facing the L5 and S1 vertebrae, respectively. It is further contemplated that cavity 29 may include openings variously disposed about the surface of implant 28.

For example, cavity 29 of implant 28 may include at least one agent including biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, biologically active agents coated onto the exterior of implant 28 and/or applied thereto for gradual release such as by blending in a bioresorbable polymer that releases the biologically active agent or agents in an appropriate time dependent fashion as the polymer degrades within the patient. Suitable biologically active agents include, for example, bone morphogenic protein (BMP) and cytokines.

Implant 28 may include one or a plurality of agent reservoirs. The agent reservoirs can be configured as drug depots with medication for pain and may include antibiotics and/or therapeutics. It is envisioned that the agent reservoirs contains active agents and may include one or a plurality of therapeutic agents and/or pharmacological agents for release, including sustained release, into the lumbo-sacral region to treat, for example, pain, inflammation and degeneration. The agents may include pharmacological agents, such as, for example, antibiotics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), and combinations thereof.

The agent may also include analgesics or anesthetics such as acetic acid derivatives, COX-2 selective inhibitors, COX-2 inhibitors, enolic acid derivatives, propionic acid derivatives, salicylic acid derivatives, opioids, opioid/nonopioid combination products, adjuvant analgesics, and general and regional/local anesthetics.

The agent may also include antibiotics such as, for example, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin.

The agent may also include immunosuppressives agents, such as, for example, steroids, cyclosporine, cyclosporine analogs, cyclophosphamide, methylprednisone, prednisone, azathioprine, FK-506, 15-deoxyspergualin, prednisolone, methotrexate, thalidomide, methoxsalen, rapamycin, leflunomide, mizoribine (Bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

In one embodiment, implant system 10 includes a plurality of implants 28. It is contemplated that employing the plurality of impants 28 can optimize the amount disc space I_{5,1} can be spaced apart such that the joint spacing dimension can be preselected. The plurality of implants 28 can be oriented in a side by side engagement, spaced apart and/or staggered.

It is envisioned that the use of microsurgical and image guided technologies may be employed to access, view and repair spinal deterioration or damage, with the aid of implant system 10. Upon completion of the procedure, the surgical instruments and assemblies are removed and the incision is closed. It is contemplated that a surgical procedure employing implant system 10 may use other instruments, such as, for example, nerve root retractors, tissue retractors, forceps, cutter, drills, scrapers, reamers, separators, rongeurs, taps, cauterization instruments, irrigation and/or aspiration instruments, illumination instruments and/or inserter instruments.

Implant system 10 may be employed for performing spinal surgeries, such as, for example, discectomy, laminectomy, fusion, laminotomy, laminectomy, nerve root retraction, foramenotomy, facetectomy, decompression, spinal nucleus or disc replacement and bone graft and implantable prosthetics including plates, rods, and bone engaging fasteners.

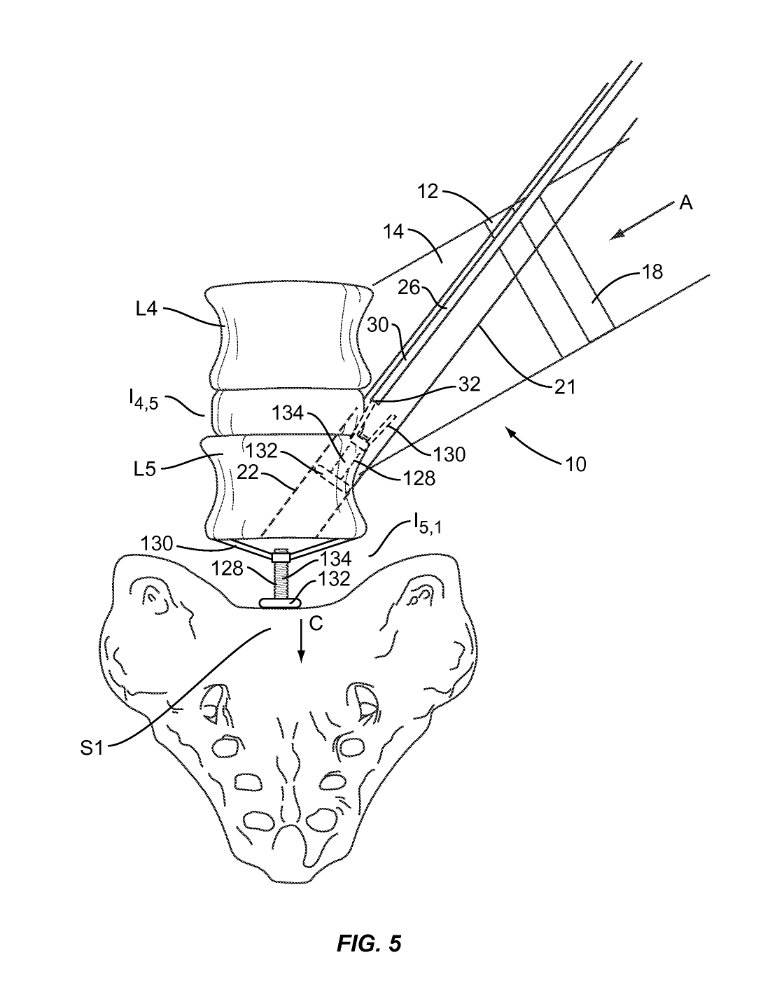

In one embodiment, as shown in FIG. 5, implant system 10, similar to that described above, includes an implant 128, which is expandable between a collapsed configuration (shown in phantom) and an expanded configuration within disc space I_{5,1}. Distal end 32 of driver 30 engages and manipulates implant 128 in the collapsed configuration to deliver implant 128 to disc space I_{5,1}. Driver 30 rotates implant 128 about bore 22 and the lower endplate of the L5 vertebrae, as described above, so that implant 128 becomes disposed in disc space I_{5,1}. As implant 128 is delivered into disc space I_{5,1}, wings 130 expand with a spring biased force or force from manipulation with driver 30 to anchor implant 128 with the L5 lower endplate. Implant 128 includes a platform 132 that engages the upper endplate of the S1 vertebrae to provide distraction of disc space I_{5,1}. Once expanded, implant 128 may be locked into place such that it cannot collapse.

Wings 130 are connected with platform 132 by a threaded bolt 134. As bolt 134 is rotated, platform 132 applies increased force against the upper S1 endplate. Platform 132 extends, in the direction shown by arrow C in FIG. 5, to provide distraction of disc space I_{5,1}. It is envisioned that bolt 134 may be manipulated with an articulating driver. It is contemplated that implant 128 creates a void in disc space I_{5,1} to maximize bone graft insertion therein. It is further contemplated that implant 128 may include an expanding butterfly wing configuration and/or fixation elements. It is envisioned that implant 128 is employed similar to implant 28 described above.

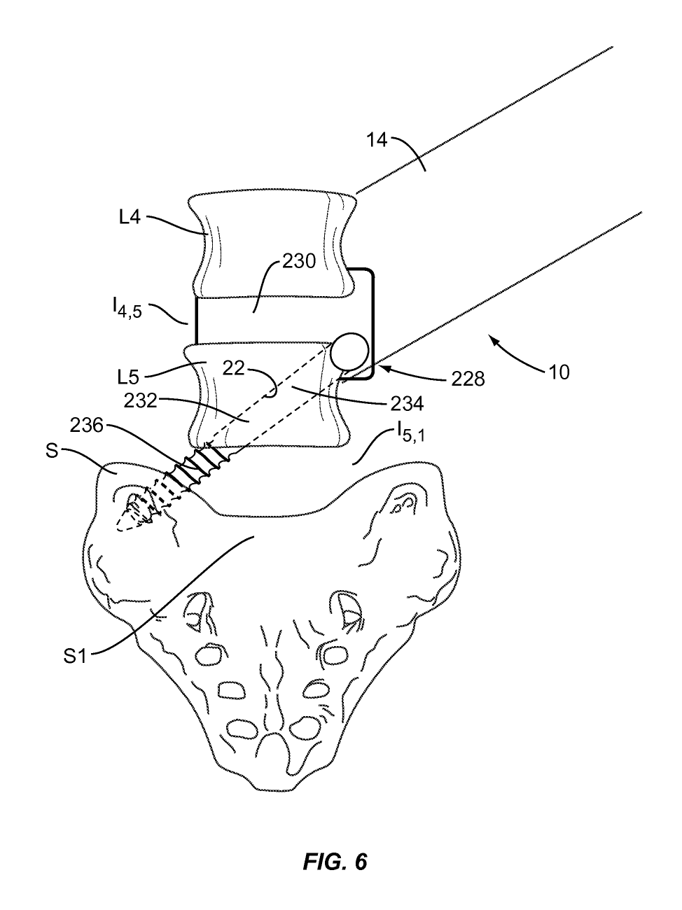

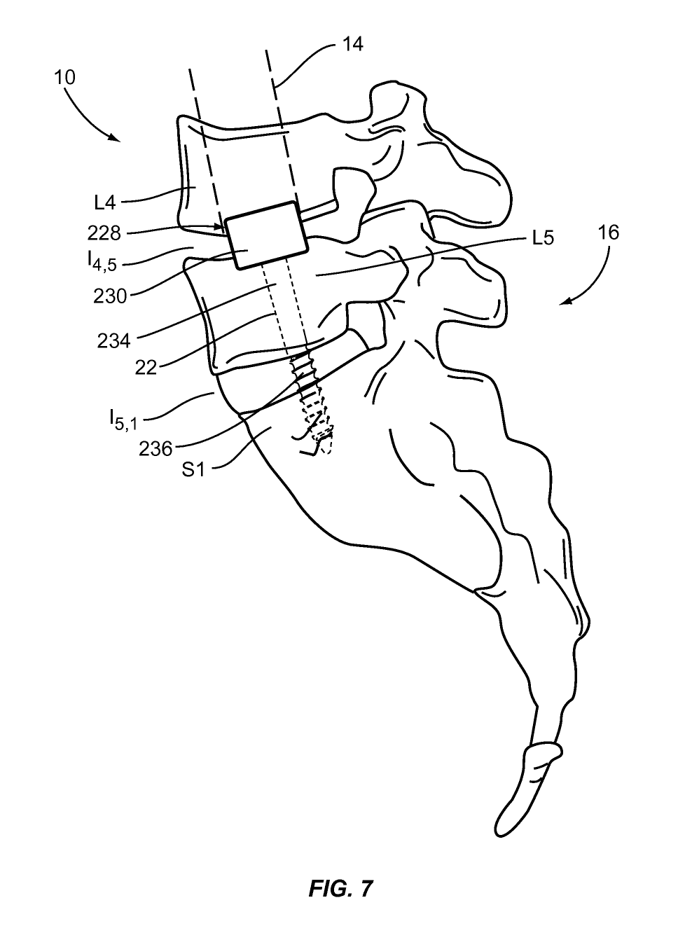

In one embodiment, as shown in FIGS. 6-7, implant assembly 10, similar to that described above, includes an implant 228 having a L4-L5 intervertebral implant 230 configured for disposal and treatment of a L4-L5 intervertebral disc space I_{4,5} and a screw 232. Screw 232 is configured to extend through bore 22 and disc space I_{5,1}, and anchor within a sacrum S of body 16. Screw 232, which is anchored within sacrum S, secures implant 230 with disc space I_{4,5} and the lumbo-sacral region thereby immobilizing the L5-S1 junction. Screw 232 has a lag portion 234 and a threaded portion 236 configured for penetrating sacrum S.

Implant 230 is delivered through passageway 14 via sleeve 21 (FIG. 3) and implanted with disc space I_{4,5}. Screw 232 is delivered through passageway 14 via sleeve 21 to bore 22, similar to implant 28 described above. Distal end 32 of driver 30 (FIGS. 3-4) engages screw 232 to deliver screw 232 through bore 22 and into disc space I_{5,1} such that threaded portion 236 is oriented for penetrating sacrum S. Screw 232 is manipulated/rotated such that threaded portion 236 penetrates and is fixed within sacrum S. Screw 232 includes a head 238 connected with implant 230 to secure implant 230 in place upon anchoring of screw 232 with sacrum S. As such, implant 230 is anchored within disc space I_{4,5}, thereby immobilizing the lumbo-sacral region and preventing back out of screw 232. It is envisioned that implant 228 is delivered similar to implant 28 described above.

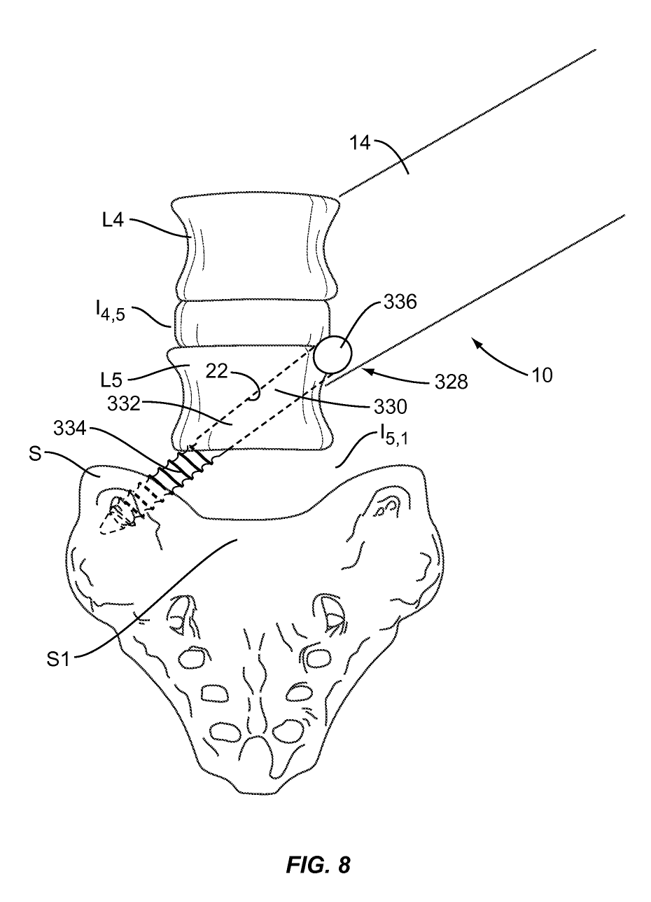

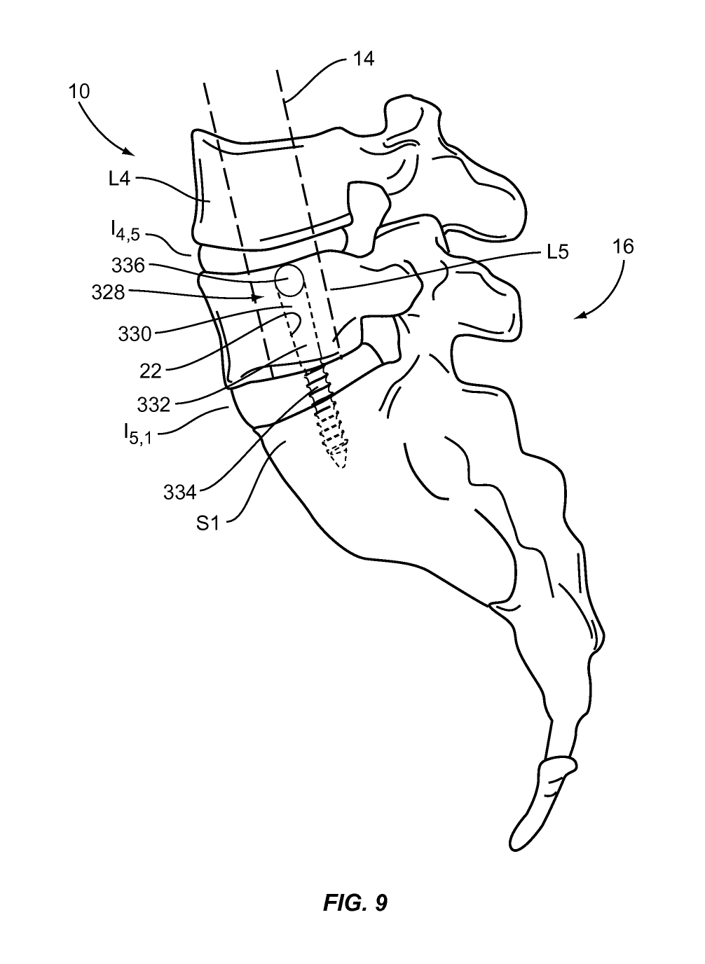

In one embodiment, as shown in FIGS. 8-9, implant assembly 10, similar to that described above, includes an implant 328 having a screw 330 configured to extend through bore 22 and disc space I_{5,1}, and anchor within a sacrum S of body 16. Screw 330 is anchored within sacrum S and through the lumbo-sacral region to immobilize the L5-S1 junction. Screw 330 has a lag portion 332 and a threaded portion 334 configured for penetrating sacrum S.

Screw 330 is delivered through passageway 14 via sleeve 21 (FIG. 3) to bore 22, similar to implant 28 described above. Distal end 32 of driver 30 (FIGS. 3-4) engages screw 330 to deliver screw 330 through bore 22 and into disc space I_{5,1} such that threaded portion 334 is oriented for penetrating sacrum S. Screw 330 is manipulated/rotated such that threaded portion 334 penetrates and is fixed within sacrum S. Screw 330 includes a head 336 that is fastened with an upper endplate and/or other regions of the L5 vertebrae to secure screw 330 in place. As such, screw 330 is anchored with the L5 vertebrae adjacent the disc space I_{4,5} and sacrum S to immobilize the lumbo-sacral region and prevent back out of screw 330. It is envisioned that implant 328 is delivered similar to implant 28 described above.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A system for treating a lumbo-sacral junction, the system comprising:
at least one dilator configured to create a passageway from a direct lateral trajectory in a body defining a longitudinal axis;
a cutting instrument configured to create a bore, oriented at angle from the longitudinal axis, in a L5 vertebrae of the body extending to a central portion of a L5-S1 intervertebral disc space of the body; and
a delivery instrument configured to deliver an implant through the bore to the central portion of the L5-S1 disc space.

2. The system for treating a lumbo-sacral junction of claim 1, wherein the at least one dilator includes a plurality of dilators employed with a direct lateral interbody surgical approach.

3. The system for treating a lumbo-sacral junction of claim 1, wherein the at least one dilator is configured for insertion with an incision created in a lateral portion of the body in a direct lateral interbody surgical approach.

4. The system for treating a lumbo-sacral junction of claim 1, further comprising a retractor defining a transverse axis, the retractor being connected with the passageway and configured for mounting with the body such that the transverse axis is disposed at a non-parallel orientation with the longitudinal axis of body.

5. The system for treating a lumbo-sacral junction of claim 1, wherein the delivery instrument includes a driver disposed at a distal end thereof, the driver being configured to engage the implant such that the implant is deliverable along the angle of the bore and rotatable relative to the bore into the central portion of the L5-S1 intervertebral disc space.

6. The system for treating a lumbo-sacral junction of claim 5, wherein the driver has a ball configuration and the implant defines a socket for receiving the ball such that the implant is deliverable and rotatable.

7. The system for treating a lumbo-sacral junction of claim 5, wherein the driver includes ball tipped tamp configuration and the implant defines a socket for receiving the ball tipped tamp such that the implant is deliverable and rotatable.

8. The system for treating a lumbo-sacral junction of claim 1, wherein the implant is expandable between a collapsed configuration and an expanded configuration within the L5-S1 intervertebral disc space.

9. The system for treating a lumbo-sacral junction of claim 8, wherein the implant includes spring-loaded, expandable wings.

10. The system for treating a lumbo-sacral junction of claim 1, wherein the implant includes a screw configured to extend through the bore and the L5-S1 intervertebral disc space, and anchor within a sacrum of the body.

11. A method for treating a lumbo-sacral junction, the method comprising the steps of:
making an incision in a lateral portion of a body, the body defining a longitudinal axis;
creating a passageway extending from the incision to adjacent a L4-L5 intervertebral disc space of the body, the passageway being disposed at a first angle from the longitudinal axis;
creating a bore in an L5 vertebrae of the body, the bore extending at a second angle from the longitudinal axis from the passageway to a central portion of an L5-51 intervertebral disc space of the body;
preparing the L5-S1 intervertebral disc space; and
delivering an implant through the bore to the central portion of the L5-S1 intervertebral disc space for treatment.

12. The method for treating a lumbo-sacral junction of claim 11, wherein the step of creating a passageway includes inserting at least one dilator with the incision.

13. The method for treating a lumbo-sacral junction of claim 11, wherein the step of creating a passageway includes mounting a retractor with the incision, the retractor defining a transverse axis such that, upon mounting, the transverse axis is disposed at a non-parallel orientation with the longitudinal axis of the body.

14. The method for treating a lumbo-sacral junction of claim 11, wherein the step of delivering an implant includes an instrument configured to deliver the implant to the central portion of the L5-S1 intervertebral disc space through the bore, the instrument including a driver disposed at a distal end thereof, the driver being configured to engage the implant such that the implant is deliverable along the angle of the bore and rotatable relative to the bore into the central portion of the L5-S1 intervertebral disc space.

15. The method for treating a lumbo-sacral junction of claim 14, wherein the driver has a ball configuration and the implant defines a socket for receiving the ball such that the implant is deliverable and rotatable.

16. The method for treating a lumbo-sacral junction of claim 15, wherein the driver includes ball tipped tamp configuration and the implant defines a socket for receiving the ball tipped tamp such that the implant is deliverable and rotatable.

17. The method for treating a lumbo-sacral junction of claim 11, wherein the implant is expandable such that the implant has a collapsed configuration within the bore and an expanded configuration within the L5-S1 intervertebral disc space.

18. The method for treating a lumbo-sacral junction of claim 11, wherein the implant includes spring loaded, expandable wings.

19. The method for treating a lumbo-sacral junction of claim 11, wherein the implant includes a screw configured to extend through the bore and the L5-S1 intervertebral disc space, and anchor within a sacrum of the body.

20. A system for treating a lumbo-sacral junction employing a direct lateral interbody fusion approach, the system comprising:
a plurality of dilators configured to create a passageway through a direct lateral trajectory in a body defining a longitudinal axis, the passageway being disposed at a first angle from the longitudinal axis;
a retractor defining a transverse axis and being connected with the passageway, the retractor being configured for mounting with the body such that the transverse axis is disposed at a non-parallel orientation with the longitudinal axis;
a cutting instrument configured to create a bore extending at a second angle from the longitudinal axis from the passageway to a central portion of a L5-S1 intervertebral disc space of the body;
a delivery instrument including a driver connected to a distal end thereof; and
an implant defining a socket that receives the driver and is engaged thereby such that the implant is deliverable along the angle of the bore and rotatable relative to the bore into the central portion of the L5-S1 intervertebral disc space.
